(19) United States
(12) Patent Application Publication
(10) Pub. No.: US 2010/0297246 A1
(43) Pub. Date: Nov. 25, 2010

(54) SILICA-BASED NANOPARTICLES AND METHODS OF STIMULATING BONE FORMATION AND SUPPRESSING BONE RESORPTIOIN THROUGH MODULATION OF NF-KB

(76) Inventors: Mervyn Neale Weitzmann, Decatur, GA (US); George Richard Beck, Decatur, GA (US); Jin-Kyu Lee, Seoul (KR)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/676,652

(22) PCT Filed: Sep. 5, 2008
(86) PCT No.: PCT/US2008/075360

§ 371 Date: Aug. 6, 2010

Related U.S. Application Data
(60) Provisional application No. 60/970,315, filed on Sep. 6, 2007.

(51) Int. Cl. A61K 9/16; C12N 5/02; C12N 5/071; A61K 33/00

(52) U.S. Cl. 424/496; 435/375; 435/366; 424/724; 977/773; 977/904

(57) ABSTRACT

Osteoporosis, is an exceedingly common malady that leads to bone fracture and results from an imbalance in the rate of osteoblastic bone formation with respect to osteoclastic bone degradation. Nanotechnology has raised exciting possibilities for the development of novel therapeutic agents. Embodiments of the disclosure provide silica-based fluorescent nanoparticles endowed with natural bone targeting capabilities and expressing potent pro-osteoblastogenic and concomitant anti-osteoclastogenic activities in vitro and the capacity to increase bone mineral density in vivo. Embodiments of the disclosure can achieve their stimulatory effects on osteoblasts, and inhibitory effects on osteoclasts, in part by suppressing NF-κB signal transduction. Embodiments of the present disclosure provide for derivatives of silica-based nanoparticles that represent a novel class of dual anti-catabolic and pro-anabolic agents that may be applicable to the amelioration of numerous osteoporotic conditions.

### CROSS-REFERENCE TO RELATED APPLICATIONS

This application claims priority to U.S. Provisional Patent Application Ser. No. 60/970,315 entitled “Silica-based nanoparticles and methods of stimulating bone formation and suppressing bone resorption, through modulation of NF-κB” filed on Sep. 6, 2007, the entirety of which is hereby incorporated by reference.

### TECHNICAL FIELD

The present disclosure is generally related to silica-based nanoparticles and their therapeutic use in modulating bone turnover.

### BACKGROUND

Osteoporosis is reaching epidemic proportions and strategies to manage this disease have centered historically on the use of antiresorptive agents design to slow further bone loss, and allow bone formation to restore bone mass. In reality, because the processes of bone resorption and bone formation are “coupled”, pharmacological suppression of bone resorption is typically observed to be accompanied by a similar decline in bone formation (McClung et al., N. Engl. J. Med. 354: 821-831. (2006)). Consequently the effect of coupling makes it particularly difficult to effectively restore lost bone mass, and antiresorptive drugs generally fail to fully prevent the occurrence of new fractures once osteoporosis is established (Reginster et al., Drugs Today (Barc.) 39: 89-101 (2003).

Recent advances have led to the development of agents capable of stimulating bone formation, with TERIPARATIDE™, a fragment of the human parathyroid hormone (hPTH), being the first of these dedicated anabolic agents to be FDA approved for the treatment of postmenopausal osteoporosis. Interestingly, the pretreatment with, or combined use of, antiresorptive agents such as bisphosphonates and TERIPARATIDE™ appears to impair the ability of parathyroid hormone to increase the bone mineral density (Delmas et al., Bone 16: 603-610 (1995); Finkelstein et al., N. Engl. J. Med. 349: 1216-1226 (2003)). The development, therefore, of anabolic agents that are more compatible with simultaneous antiresorptive therapy, or that themselves exhibit dual anabolic and anticatabolic activities, would be valuable therapeutic assets in the fight against osteoporosis, especially those severe forms where aggressive therapy may be indicated.

Nanotechnology is a multidisciplinary field involving the development of engineered “devices” at the atomic, molecular and macromolecular level, in the nanometer size range (Navalakhe & Nandedkar, Ind. J. Exp. Biol. 45: 160-165 (2007); Sahoo et al., Nanomedicine 3: 20-31 (2007)). Applications of nanotechnology to medicine and physiology typically involve materials and devices designed to interact with the body at subcellular (molecular) scales with a high degree of specificity. This can be potentially translated into targeted cellular and tissue-specific clinical applications designed to achieve maximal therapeutic efficacy with minimal side effects (Sahoo et al., Nanomedicine 3: 20-31 (2007)).

One material used in the application of nanotechnology in medicine is silica. Silica-based nanoparticles appear to have good biocompatibility as they are generally thought to be non-toxic in vivo. Dietary silica is generally presumed safe in humans and no adverse effects are observed in rodents at doses as high as 50,000 ppm (Martin, J. Nutr. Health Aging 11:94-97 (2007)). Silica is used extensively as a food additive, and as inactive filler in drugs and vitamins. Being the second most prevalent element after oxygen (Martin, J. Nutr. Health Aging 11: 94-97 (2007)), silica is abundant and cheap.

Chemically, silica is an oxide of silicon, (silicon dioxide), and orthosilicic acid is the form predominantly absorbed by humans and is found in numerous tissues including bone, tendons, aorta, liver and kidney. Silica deficiency leads to detrimental effects on the skeleton including skull and peripheral bone deformities, poorly formed joints, defects in cartilage and collagen, and disruption of mineral balance in the femur and vertebrae (Martin, J. Nutr. Health Aging 11, 94-97 (2007)). Silicon has also been suggested to play a physiological role in bone formation (Seaborn & Nielsen, Biol. Trace Element Res. 89: 239-250 (2002)) although the action of silicon on bone turnover and structure is presently not clear.

The dissolution of bone structure leads to osteoporosis, a condition that predisposes the skeleton to fracture. Bone fractures incur monumental health care costs to patients and society. Total fractures in 2005 exceeded 2 million, costing nearly $17 billion. The aging of the U.S. population will likely lead to greater prevalence of osteoporosis and annual fractures and costs are projected to rise by almost 50% by 2025 (Burge et al., J. Bone Miner. Res. 22: 465-475 (2007)). Hip fractures may cause prolonged or permanent disability and almost always require hospitalization and major surgery. Spinal or vertebral fractures have serious consequences, including loss of height, severe back pain, and deformity.

The skeleton is a dynamic organ that undergoes continuous regeneration involving the resorption (breakdown) of old bone by osteoclasts and its resynthesis by osteoblasts. Osteoclast precursors are derived from cells of the monocytic lineage and physiological osteoclast renewal is regulated principally by action of the key osteoclastogenic cytokine Receptor Activator of NF-κB Ligand (RANKL), in the presence of permissive levels of the trophic factor Macrophage Colony Stimulating factor (M-CSF) (Teitelbaum, Science 289: 1504-1508 (2000)). Osteoclast precursors differentiate into preosteoclasts expressing Tartrate Resistant Acid Phosphatase (TRAP), which fuse into multinucleated mature bone-resorbing osteoclasts.

Osteoblasts, the cells that synthesize bone are derived from pluripotent mesenchymal stem cells (Aubin & Triffitt, Mesenchymal Stem Cells and Osteoblast Differentiation, Vol. 1, 2nd edn. (San Diego, Academic press) (2002)). Secreted and intracellular mediators promote the differentiation and survival of osteoblasts including Transforming Growth Factor beta (TGFβ), bone morphogenic proteins (BMPs)−2, −4, −6 and −7, and insulin like growth factor I (IGF-I) (Gilbert et al., Endocrinology 141: 3956-3964 (2000)). Two critical events in osteoblast differentiation are the upregulation of the transcription factors Runt related transcription factor-2 (Runx2) (Ducy et al., Cell 89: 747-754 (1997)) and Osterix (Nakashima et al., Cell 108: 17-29 (2002).

The NF-κB signal transduction pathway is recognized as critical for osteoclast development and function (Boyce et al., Bone 25: 137-139 (1999); Franzoso et al., Genes Dev 11: 3482-3496 (1997)). Double knockout (KO) of p50 and p52 NF-κB subunits leads to defective osteoclast differentiation, and to osteopetrosis (high bone mass) (lotsova et al., Nat. Med. 3: 1285-1289 (1997)). NF-κB antagonists prevent bone destruction by suppressing osteoclast activity (Hall et al., Biochem. Biophys. Res. Commun. 207: 280-287 (1995)) in vitro, and in animal models of postmenopausal osteoporosis (Strait et al., Int. J. Mol. Med. 21: 521-525 (2008)) rheumatoid arthritis (Dai et al., J. Biol. Chem. 279: 37219-37222 (2004)), and in models of multiple myeloma in vitro (Feng et al., Blood 109: 2130-2138 (2007)).

In contrast to osteoclasts, it has been reported (Li et al., J. Bone Miner. Res. 22: 646-655 (2007)) that induction of NF-κB by TNFα or other cytokines potently suppresses osteoblast differentiation in vivo and in vitro in part by antagonizing Smad activation by TGFβ and/or BMP through induction of NF-κB (Li et al., J. Bone Miner. Res. 22: 646-655 (2007); Eliseev et al., Exp. Cell Res. 312: 40-50 (2006)). Furthermore, pharmacological suppression of NF-κB blocks TNFα-induced osteoblast suppression in vitro (Li et al., J. Bone Miner. Res. 22: 646-655 (2007)) while NF-κB suppression in MC3T3 preosteoblasts and in primary mouse bone marrow stromal cells dramatically enhances mineralization (Li et al., J. Bone Miner. Res. 22: 646-655 (2007)) demonstrating that endogenous basal NF-κB signal transduction antagonizes osteoblast differentiation and mineralization.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a digital image that illustrates that NP1 nanoparticles dose-dependently inhibit RANKL-induced osteoclast formation. TRAP stained osteoclasts were photographed under light microscopy at 100× magnification.

FIG. 2 illustrates that NP1 nanoparticles suppress osteoclastic differentiation of RAW264.7 cells in vitro. Mature multinucleated (≧3 nuclei) TRAP osteoclasts were quantified in NP1 treated cultures. All data points represent average±S.D. of 4 replicate wells and 3 or more independent experiments. The TRAP stained osteoclast cultures (right hand image) are from a representative experiment.

FIG. 3 is a graph illustrating that NP1 nanoparticles suppress osteoclastic differentiation of primary monocytes in vitro. The graph shows that NP1 nanoparticles dose-dependently inhibit differentiation of primary monocytes into osteoclasts. All data points represent average±S.D. of 4 replicate wells.

FIG. 4 is a graph illustrating that NP1 suppresses early differentiation of RAW264.7 cells into osteoclasts. RAW264.7 cells were treated with RANKL, and NP1 (50 μg/ml) added at day 1, 3 or 5 of culture. Cultures were TRAP stained at day 7 and mature osteoclasts then quantified.

FIGS. 5A-5E are graphs illustrating that the nanoparticle NP1 does not affect the viabilities of a variety of cultured cell lines.

FIG. 6 illustrates that NP2 nanoparticles suppress osteoclastogenesis in vitro. The graph shows that NP2 dose-dependently inhibits RANKL-induced osteoclast formation. All data points represent average±S.D. of 4 replicate wells and 3 or more independent experiments. The digital photograph shows TRAP stained osteoclast cultures from a representative experiment.

FIG. 7 are digital images illustrating NP1 dose-dependently induces mineralization nodules in MC3T3 cultures. Stained with alizarin red-S at 11 days.

FIG. 8 illustrates a Northern blot showing that NP1 dose-dependently induces expression of the characteristic osteoblastic gene products bone sialoprotein, osteocalcin and osteopontin in MC3T3 cells.

FIG. 9 illustrates a Western blot showing NP1 (50 μg/ml for 18 hr) stimulated expression of Runx2 and rescue of TNFα-induced suppression of Runx2 in MC3T3 cells.

FIG. 10 are digital images illustrating NP2 induces osteoblastic differentiation of MC3T3 cells analogous to NP1. Alizarin red-S staining.

FIG. 11 illustrates a time-course for NP1 internalization into MC3T3 preosteoblastic cells. NP1 was at 60 μg/ml. Digital images were under bright field (lower panels) and fluorescent microscopy (upper panels).

FIG. 12 illustrates NP1 and NP2 internalization in RAW267.4 osteoclast precursors (white arrows) and mature multinucleated osteoclasts (solid arrows). Left panels show TRAP-stained cultures under bright field; middle panels show fluorescence microscopy images, and right panels show bright field and fluorescence images merged.

FIG. 13 shows fluorescent confocal microscopy images of nanoparticle cellular localization: MC3T3 cells were treated with nanoparticles 60 μg/ml for 1 hour, and with lysomal tracker, and endosome tracker. NP1, endosomes, and lysosome fluorescence images were also merged, thereby showing co-localization of NP1 and endosomes (white arrow). Images were captured by a Zeiss LSM 510 META point scanning laser confocal microscope.

FIGS. 14A-14C are transmission electron micrographs (TEM) of MC3T3 cells treated with: (FIG. 14A) NP3, a metal core variant of NP1; (FIG. 14B) without NP1 treatment; and (FIG. 14C) a high resolution TEM of NP3 treated MC3T3 cells showing SiO₂-coated NP3 (yellow arrow), and uncoated NP3 cores (red arrows). Inset: digital image showing monodispersed NP3 in solution.

FIGS. 15A-15C are graphs illustrating NP1 nanoparticle suppression of NF-κB activation. NP1 dose-dependently suppresses TNFα-induced NF-κB activity in MC3T3 cells (FIG. 15A) transfected with an NF-κB-responsive luciferase reporter. NP1 does not suppress TNFα-induced NF-κB activity in HEK293 cells (FIG. 15B). Over-expression of p65 NF-κB subunit prevents NP1-induced suppression of NF-κB luciferase activity in MC3T3 cells (FIG. 15C). All data points represent average±S.D. of 4 replicate wells and at least two independent experiments.

FIG. 16 is a radiograph showing that NP1 blocks the basal and TNFα-induced proteasomal cleavage of NF-κB precursor p105 into its active p50 subunit.

FIGS. 17A and 17B illustrate that NP1 nanoparticles do not modulate Smad, or Wnt pathways, or reactive oxygen species (ROS). As show in FIG. 17C, MC3T3 cells were loaded with DCF-DA and NP1 and ROS examined by fluorescence microscopy (middle panel). Top panel shows cells under light microscopy, and the bottom panel shows NP1 fluorescence. FIG. 17D shows photographs of a northern blot showing the capacity for NP1 to upregulate basal osteocalcin and osteopontin gene expression in MC3T3 cells, but not in RAW264.7 and NIH3T3 cells.

FIG. 18 shows fluorescent digital images illustrating that NP1 and NP2 nanoparticles directly bind to bone surfaces. Dentine slices, devitalized bovine cortical bone slices, Biocoat Osteologic discs and hydroxyapatite (HA) crystals were incubated with NP1 or NP2 for 2 hr and washed extensively before examination under light and fluorescence microscopy. Images photographed at 200× magnification.

FIG. 19 illustrates NP1 binding to BIOCOAT™ calcium phosphate analog, but not to the quartz substrate.

FIG. 20 illustrates a series of scanning electron photomicrographs of control and NP1 treated dentine slices. Nanoparticles (white dots) are indicated by solid arrows.

FIGS. 21A and 21B are graphs illustrating that NP4 nanoparticles can increase bone mass in vivo. Female C57BL6 mice, eight weeks of age, were injected intraperitoneal with 50 mg/Kg of NP4 in PBS or vehicle (PBS) alone, once per week. BMD was measured by DXA every two weeks up to 6 weeks at: the lumbar spine (FIG. 21A), or femur (FIG. 21B) (average of left and right femur from each mouse). Data represents average±SEM, n=9/mice group; Repeated measures ANOVA. *p≦0.005, **p≦0.001.

FIGS. 22A and 22B show representative 20 μm micro-CT reconstructions of vehicle and NP4 treated vertebrae (FIG. 22A) and femurs (FIG. 22B).

FIGS. 23A and 23B are graphs illustrating vertebral (FIG. 23A) and femur (FIG. 23B) structural indices computed by micro-CT for vehicle and NP4 treated mice: TV: trabecular volume; BV: bone volume, CD: connectivity density, SMI: structural model index, Tb.N: trabecular number, Tb.Th: trabecular thickness, Tb.Sp: trabecular separation, TV.D: trabecular volume density, BV.D: bone volume density. N=7 mice per group.

FIG. 24 is a graph illustrating biochemical indices of bone formation in vivo.

FIG. 25 schematically illustrates the synthesis of fluorescent silica nanoparticles: SiO_{2 (RhB).}

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a nanoparticle” includes a plurality of nanoparticles. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially” of or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Abbreviations

RANKL, Receptor Activator of NF-κB Ligand; M-CSF, Macrophage Colony Stimulating factor; TRAP, Tartrate Resistant Acid Phosphatase; NP, nanoparticle; TNF, tumor necrosis factor; DXA, Dual-energy X-ray Absorptiometry; BMD, bone mineral density; TGFβ, transforming growth factor beta; BMP, bone morphogenetic protein; micro (μ)-CT. micro-computed tomography; ROS, reactive oxygen species; RhB, rhodamine B; Osx, osterix.

### DEFINITIONS

The term “osteoblast” as used herein refers to cells involved in both endochondral and intramembranous ossification, and which are the specialized cells in bone tissue that make matrix proteins resulting in the formation of new bone. These bone-forming cells are derived from mesenchymal osteoprogenitor cells. They form an osseous matrix in which they may become enclosed as an osteocyte. They are capable of differentiating to other lineages such as adipocytes, chondrocytes and muscle.

The term “osteoclast” as used herein refers to cells used in endochondral ossification. They dissolve calcium previously stored away in bone and carry it to tissues whenever needed. Thus, while osteoblasts are associated with new bone growth, osteoclasts are associated with bone resorption and removal.

The term “osteogenesis,” as used herein refers to the proliferation of osteoblasts and growth of bone mass (i.e., synthesis and deposit of new bone matrix). Osteogenesis also refers to differentiation or transdifferentiation of progenitor or precursor cells into bone cells (i.e., osteoblasts). Progenitor or precursor cells can be pluripotent stem cells such as, e.g., mesenchymal stem cells. Progenitor or precursor cells can be cells pre-committed to an osteoblast lineage (e.g., pre-osteoblast cells) or cells that are not pre-committed to an osteoblast lineage (e.g., pre-adipocytes or myoblasts).

The term “pharmaceutically acceptable carrier” as used herein refers to a diluent, adjuvant, excipient, or vehicle with which a heterodimeric probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the heterodimeric probe and pharmaceutically acceptable carriers can be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The terms “core” or “nanoparticle core” as used herein refers to the inner portion of nanoparticle. A core can substantially include a single homogeneous monoatomic or polyatomic material. A core can be crystalline, polycrystalline, or amorphous, metallic or non-metallic. A core may be “defect” free or contain a range of defect densities. In this case, “defect” can refer to any crystal stacking error, vacancy, insertion, or impurity entity (e.g., a dopant) placed within the material forming the core. Impurities can be atomic or molecular.

While a core may herein be sometimes referred to as “crystalline”, it will be understood by one of ordinary skill in the art that the surface of the core may be polycrystalline or amorphous and that this non-crystalline surface may extend a measurable depth within the core. The core-surface region optionally contains defects. The core-surface region will preferably range in depth between one and five atomic-layers and may be substantially homogeneous, substantially inhomogeneous, or continuously varying as a function of position within the core-surface region.

Nanoparticles of the disclosure may comprise a “coat” of a second material that surrounds the core. A coat can include a layer of material, either organic or inorganic, that covers the surface of the core of a nanoparticle. A coat may be crystalline, polycrystalline, or amorphous and optionally comprises dopants or defects.

A coat may be “complete”, indicating that the coat substantially or completely surrounds the outer surface of the core (e.g., substantially all surface atoms of the core are covered with coat material). Alternatively, the coat may be “incomplete” such that the coat partially surrounds the outer surface of the core (e.g., partial coverage of the surface core atoms is achieved). In addition, it is possible to create coats of a variety of thicknesses, which can be defined in terms of the number of “monolayers” of coat material that are bound to each core. A “monolayer” is a term known in the art referring to a single complete coating of a material (with no additional material added beyond complete coverage). For certain applications, coats may be of a thickness between about 1 and 10 monolayers, where it is understood that this range includes non-integer numbers of monolayers. Non-integer numbers of monolayers can correspond to the state in which incomplete monolayers exist. Incomplete monolayers may be either homogeneous or inhomogeneous, forming islands or clumps of coat material on the surface of the nanoparticle core. Coats may be either uniform or non-uniform in thickness. In the case of a coat having non-uniform thickness, it is possible to have an “incomplete coat” that contains more than one monolayer of coat material. A coat may optionally comprise multiple layers of a plurality of materials in an onion-like structure, such that each material acts as a coat for the next-most inner layer. Between each layer there is optionally an interface region. The term “coat” as used herein describes coats formed from substantially one material as well as a plurality of materials that can, for example, be arranged as multi-layer coats.

It will be understood by one of ordinary skill in the art that when referring to a population of nanoparticles as being of a particular “size”, what is meant is that the population is made up of a distribution of sizes around the stated “size”. Unless otherwise stated, the “size” used to describe a particular population of nanoparticles will be the mode of the size distribution (i.e., the peak size). By reference to the “size” of a nanoparticle is meant the length of the largest straight dimension of the nanoparticle. For example, the size of a perfectly spherical nanoparticle is its diameter.

The term “nanoparticle” as used herein refers to a particle having a diameter of about 1 to about 1000 nm. Similarly, by the term “nanoparticles” is meant a plurality of particles having an average diameter of about 1 to about 1000 nm.

The term “pharmaceutically acceptable” as used herein refers to a compound or combination of compounds that while biologically active will not damage the physiology of the recipient human or animal to the extent that the viability of the recipient is comprised. Preferably, the administered compound or combination of compounds will elicit, at most, a temporary detrimental effect on the health of the recipient human or animal is reduced.

The preparation of a pharmacological composition that contains active ingredients dissolved or dispersed therein is well understood in the art and need not be limited based on formulation. Typically such compositions are prepared as injectables, either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

The term “stem cell,” as used herein refers to any self-renewing pluripotent cell or multipotent cell or progenitor cell or precursor cell that is capable of differentiating into multiple cell types. Stem cells suitable for use in the methods of the present invention include those that are capable of differentiating into cells of osteoblast lineage, e.g., osteoblasts and pre-osteoblast cells. Mesenchymal stem cells (MSC) are capable of differentiating into the mesenchymal cell lineages, such as bone, cartilage, adipose, muscle, stroma, including hematopoietic supportive stroma, and tendon, and play important roles in repair and regeneration. MSCs are identified by specific cell surface markers which are identified with unique monoclonal antibodies, as described in, for example, U.S. Pat. No. 5,643,736.

The term “differentiate” or “differentiation,” as used herein refers to the process by which precursor or progenitor cells (i.e., stem cells) change phenotype to become specific cell types, e.g., osteoblasts. Differentiated cells can be identified by their patterns of gene expression and cell surface protein expression. Typically, cells of an osteoblast lineage express genes such as, for example, alkaline phosphatase, collagen type I, bone sialoprotein, osteocalcin, and osteoponin. Typically, cells of an osteoblast lineage express bone specific transcription factors such as, for example, Cbfa1/Runx2 and Osx.

The terms “increase’ and “decrease’ as used herein refer to a change in a pre-existing status of a cell or physiological condition.

### DESCRIPTION

### Silica-Based Nanoparticles Having Effects on Bone Anabolism and Catabolism

The present disclosure encompasses the use of silica-based nanoparticles for the delivery of the silica to cells of an animal or human subject, where the cells are responsible for maintaining the status of osseous material. Nanoparticles suitable for use in the embodiments of the disclosure may comprise a metallic core particle such as, but not limited to, cobalt-ferrous nanoparticle core and a silicaceous shell. It is anticipated that nanoparticles for use in the methods of the present disclosure may further comprise a protective polymeric coat such as, but not limited to, a polyethylene glycol (PEG) coat, polyvinylpyrrolidone (PVP) coat, a PTMA (N-trimethoxysilylpropyl-N,N,N-trimethylammoniurn chloride) coat, a PMP [3-(trihydroxysilyl)propyl]methylphosphonate] coat, and the like, or a combination thereof. In addition, embodiments of the present disclosure may further comprise a label for monitoring the absorption and location of the nanoparticles when they are delivered to an isolated cell or animal or human subject. Silica-based nanoparticles of particular use in the methods of the disclosure are fully described in Yoon et al., Angew. Chem. Int. Ed. 44: 1068-1071 (2005), which is incorporated herein by reference in its entirety. It is to be understood that any nanoparticle having surface silica-based coatings may be of use in the methods of the disclosure, providing they increase bone formation and/or decrease bone removal, and/or increase the differentiation of stem or progenitor cells. A brief description of a method of synthesis of such nanoparticles is in Example 1, below. Silica-based nanoparticles suitable for use in the embodiments of the present disclosure, may be of a type that present on the surface thereof a silicaceous layer, or partial layer. Examples of such nanoparticles are given in Table 1, below, but it will be understood that other forms of the nanoparticles are possible, incorporating variations in the core particle, or providing a nanoparticle that does not include a metallic core but is predominantly or entirely silicaceous. It is also understood that the nanoparticles of the disclosure may include a detectable moiety such as, but not limited to, a fluorescent label, a radiolabel and the like that may be used to detect the nanoparticles within an animal or human subject, or to monitor the passage of the nanoparticles into a cell, or the locality of the particles once in the cell.

TABLE 1
Nanoparticles

| Nanoparticle Designation | Structure^{a} |
|---|---|
| NP1 | SiO₂(RhB) |
| NP2 | SiO₂(RhB)-PEG |
| NP3 | MNP-SiO₂(RhB) |
| NP4 | MNP-SiO₂(RhB)-PEG |

^{a}MNP: Magnetic Nanoparticle (The metal core) RhB: Rhodamine B derivative (The fluorescent dye) PEG: Polyethylene glycol (the surface derivative)

### NP1 Suppresses Osteoclastogenesis In Vitro.

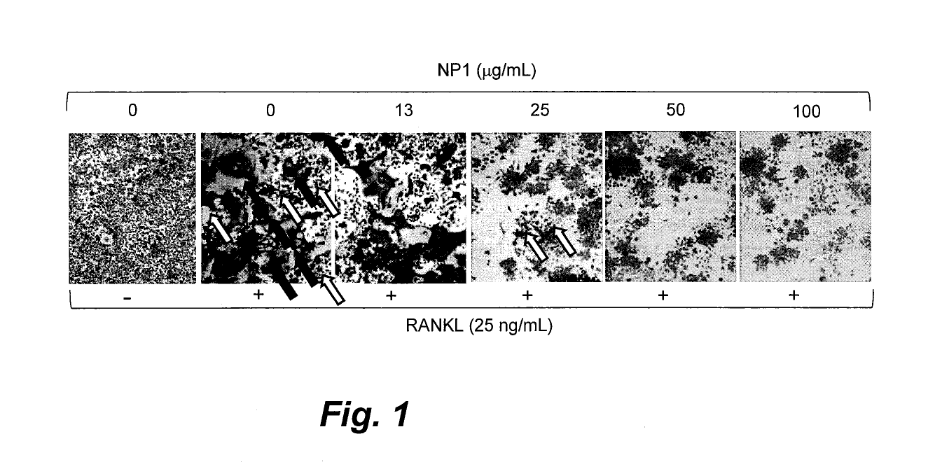

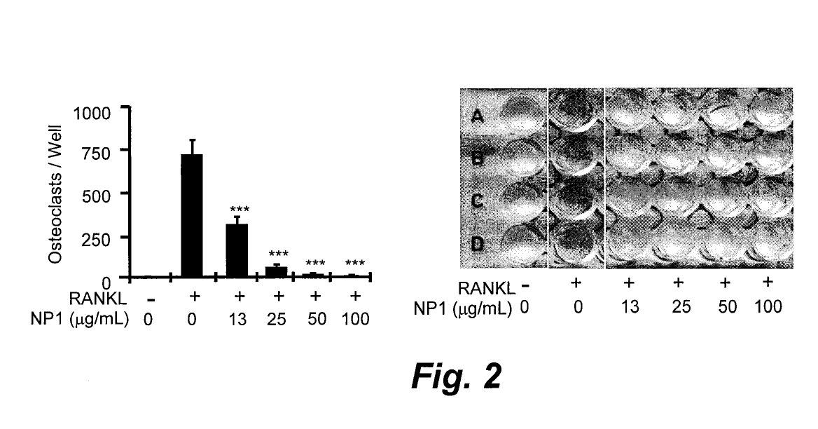

To demonstrate that silica-based nanoparticle NP1 has any inherent action on osteoclastogenesis, osteoclasts were generated in vitro by exposing RAW264.7 cells, a monocytic cell line, with RANKL in the presence of a range of NP1 concentrations from 13-100 μg/ml. Cultures were TRAP stained and photographed under light microscopy 7 days later, as shown in FIG. 1. RANKL alone stimulated the formation of large numbers of mononucleated TRAP⁺ preosteoclasts (white arrows) that fused into giant multinucleated TRAP+ mature osteoclasts (solid arrows). Addition of NP1 significantly, and dose-dependently, reduced the formation of both osteoclasts and preosteoclasts. These data were quantified by counting mature multinucleated 3 nuclei) TRAP⁺ osteoclasts, as shown in FIG. 2A, and the overall effect of the NP1 dose on total TRAP expression is shown in FIG. 2B.

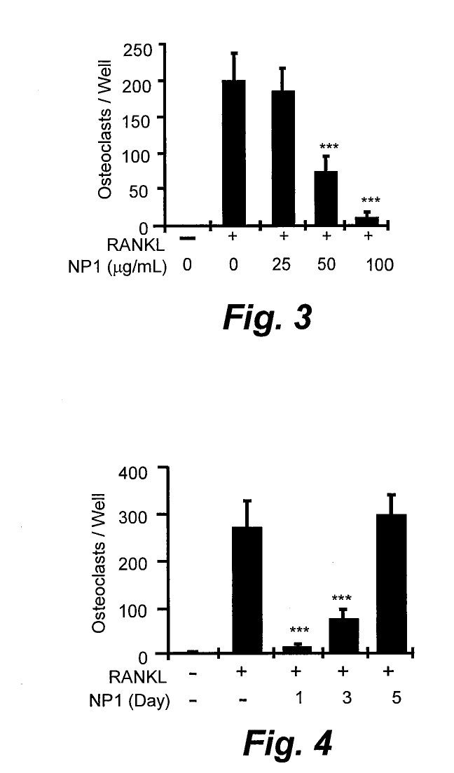

To demonstrate the capacity of NP1 to suppress osteoclastogenesis from bona fide primary mouse monocytes, osteoclasts were cultured from mouse splenic macrophages treated with RANKL and M-CSF and subjected to a range of NP1 concentrations. As with RAW264.7 cells, NP1 dose-dependently suppressed primary monocyte differentiation into osteoclasts, as shown in FIG. 3.

Osteoclasts themselves form by fusion of TRAP⁺ mononucleated preosteoclasts into multinucleated mature osteoclasts. The results as shown in FIGS. 1 and 3 show that NP1 suppressed the differentiation of RAW264.7 cells and primary monocytes into TRAP⁺ mononucleated preosteoclasts, although an effect on differentiation (fusion or osteoclast viability) may not be excluded. To examine the mechanism of NP1 action in more detail, RAW264.7 cells were cultured with RANKL, and NP1 was added at days 1, 3, or 5 of the 7 day culture period. Cultures were then stained with TRAP at day 7 and the mature osteoclasts quantified (see FIG. 4). NP1 was found to specifically suppress early differentiation of monocytic precursors into TRAP⁺ preosteoclasts (days 1-3), rather than the later fusion steps that occur at days 4 and 5 of culture period.

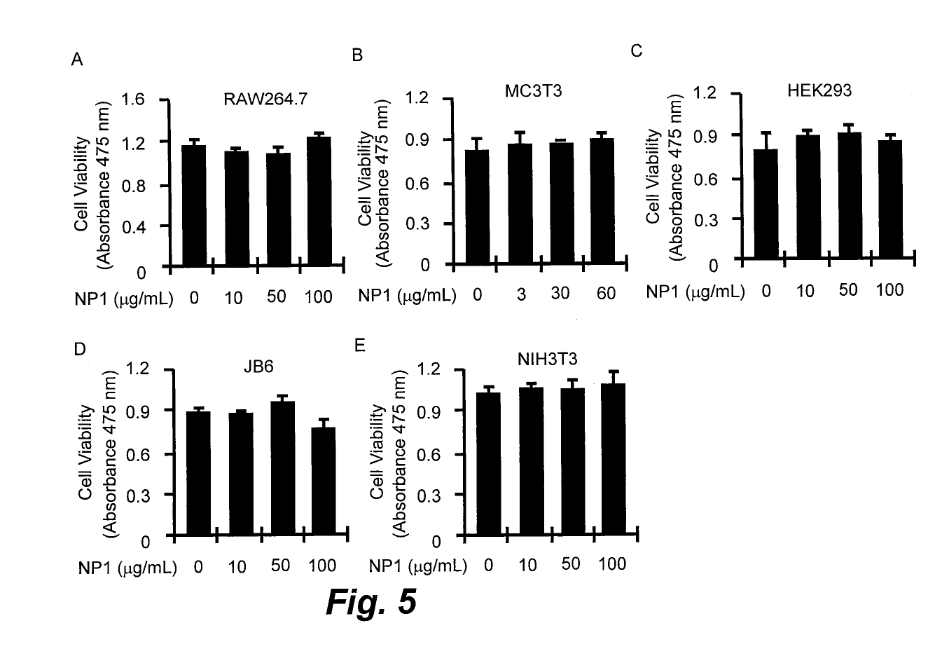

As shown in FIG. 5A, XTT (2,3-bis(2-methoxy-4-nitro-5-sulfophenyl)-5-[(phenylamino)carbonyl]-2H-tetrazolium hydroxide) viability assays indicated that NP1 does not directly alter cell viability or proliferation of non-RANKL treated monocytic cells, indicating that the primary action on NP1 is to block RANKL-induced differentiation of monocytes along the osteoclast-lineage, rather than mediating a generalized toxic effect. Additionally, NP1 also had no adverse effects on the viability of MC3T3 pre-osteoblastic cells, HEK293 kidney cells, JB6 epidermal cells or NIH3T3 mouse fibroblasts, as shown in FIGS. 5B-5E, respectively, even when treated with NP1 for up to 10 days.

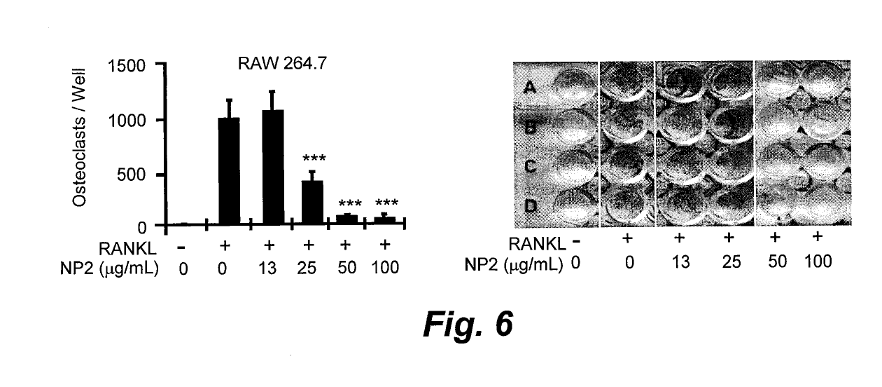

A modification that has demonstrated effectiveness in increasing circulating nanoparticle half-life, and reducing plasma clearance in vivo, involves surface derivatization with polyethylene glycol (PEG) as described by Gabizon & Martin. Drugs 54 Suppl 4: 15-21 (1997), which is incorporated herein by reference in its entirety). PEG also facilitates cytoskeletal transport by reducing steric and adhesive hindrances (see, for example, Suh et al., 2007, incorporated herein by reference in its entirety). To create, therefore, an NP1-derivative more suitable for in vivo applications nanoparticle NP2 (Table 1), a PEGylated variant of NP1, was generated. As with NP1, NP2 dose-dependently suppressed osteoclast formation, as shown in FIGS. 6A-6B.

### NP1 Stimulates Osteoblast Differentiation and Mineralization In Vitro.

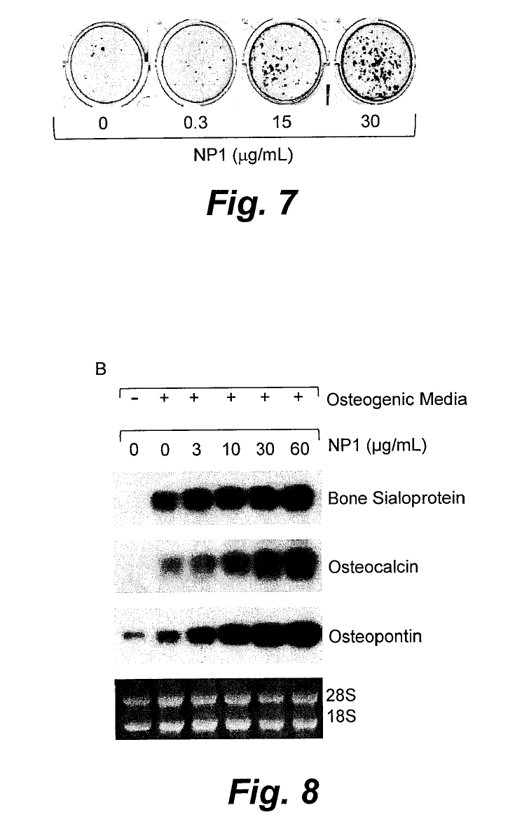

The preosteoblastic cell line MC3T3 (Sudo et al., J. Cell Biol. 96: 191-198 (1983), incorporated herein by reference in its entirety) was cultured in permissive osteogenic medium containing ascorbic acid and β-glycerophosphate in the presence of a range of NP1 concentrations. In osteogenic medium, MC3T3 cells typically differentiate spontaneously into mineralizing osteoblasts over a period of 21 days. However, mineralization nodules were readily detectable, following alizarin red-S staining, after only 11 days of culture in the presence of NP1, as shown in FIG. 7.

To validate osteoblast differentiation the expression of characteristic osteoblast genes were examined by Northern blot hybridization. NP1 dose-dependently upregulated the expression, by 7 days of culture, of key osteoblastic genes in MC3T3 cells, including bone sialoprotein, osteocalcin, and osteopontin (FIG. 8).

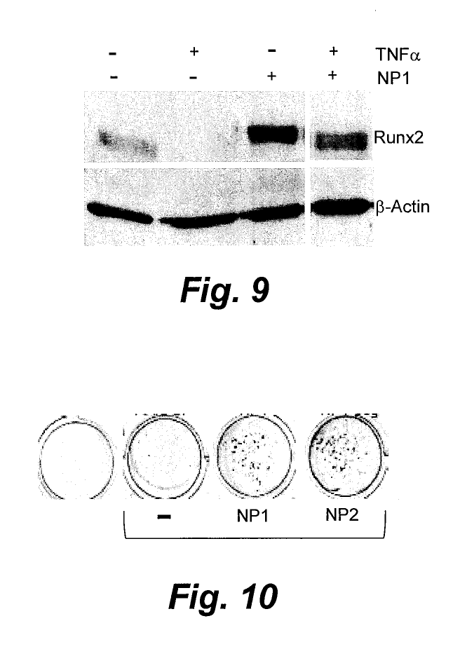

Runx2 is a critical osteoblastic transcription factor necessary for the differentiation of osteoblasts. Using western blots analysis, as shown in FIG. 9, it was found that NP1 potently stimulated Runx2 gene expression and prevented the suppression of Runx2 by TNFα (a known physiological inhibitor of osteoblast differentiation and Runx2 expression (Gilbert et al., Endocrinology 141: 3956-3964 (2002); Li et al., J. Bone Miner. Res. 22, 646-655 (2007), which are incorporated herein by reference in their entireties.

PEGylated NP1 (i.e. NP2) also promotes osteoblast differentiation and mineralization in vitro, as is shown in FIG. 10. The combined data show that NP1 dose-dependently enhances differentiation of preosteoblastic cells into mineralizing osteoblasts.

### NP1 Rapidly Enters MC3T3 and RAW264.7 Cells Through Endosomal Transport.

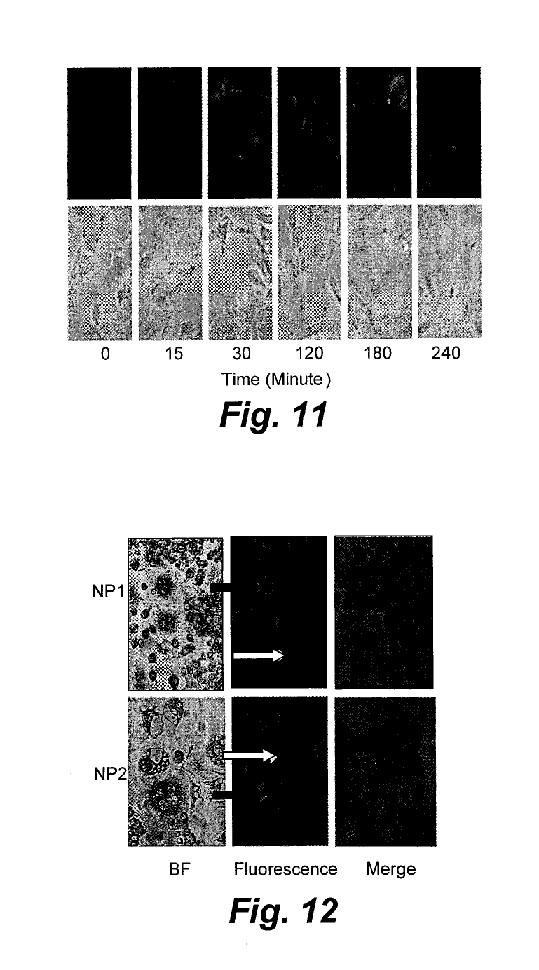

To investigate the mechanisms by which silica nanoparticles may be internalized into cells and their intracellular destination upon entry, the intense fluorescence of NP1 and NP2, a property of the fluorescent dye RhB incorporated into their shells, was used. A time-course for NP1 incorporation into MC3T3 cells, therefore, was performed by photographing MC3T3 cells exposed to NP1 at different time intervals from 15 mins to 4 hours. NP1 was rapidly internalized with intracellular fluorescence observed by 15 mins of exposure, and maximum fluorescence intensity was observed by 30 minutes, as shown in FIG. 11.

Both NP1 and NP2 were incorporated into osteoclast precursors (white arrows) and mature multinucleated osteoclasts (solid arrows) as shown in FIG. 12. In both MC3T3 and RAW264.7 cells, nanoparticles were specifically associated with the cytoplasm and excluded from nuclei.

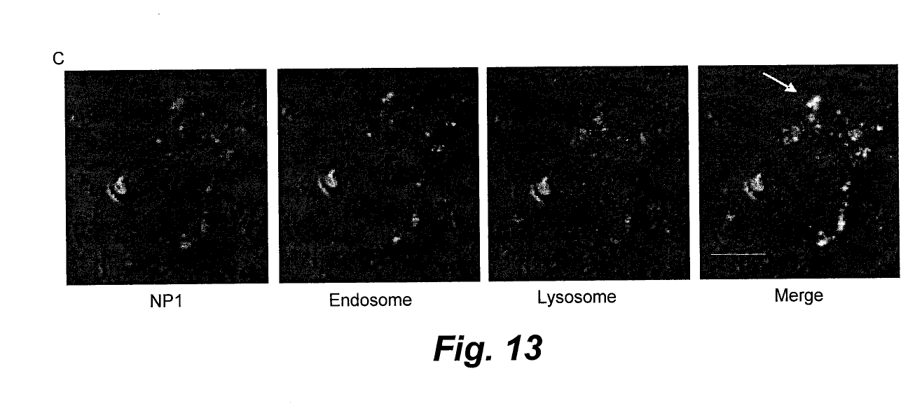

As is illustrated in FIG. 13, to further investigate NP1 subcellular localization, MC3T3 cells were loaded with NP1 for 1 hour followed by incubation with lysomal tracker (LYSOTRACKER™, Invitrogen Molecular Bioprobes) and endosome tracker (Transferrin-GFP, Invitrogen), and examined by confocal microscopy. Fluorescent images were merged and demonstrated colocalization between NP1 and endosomes (white arrow) (see FIG. 13, fourth panel).

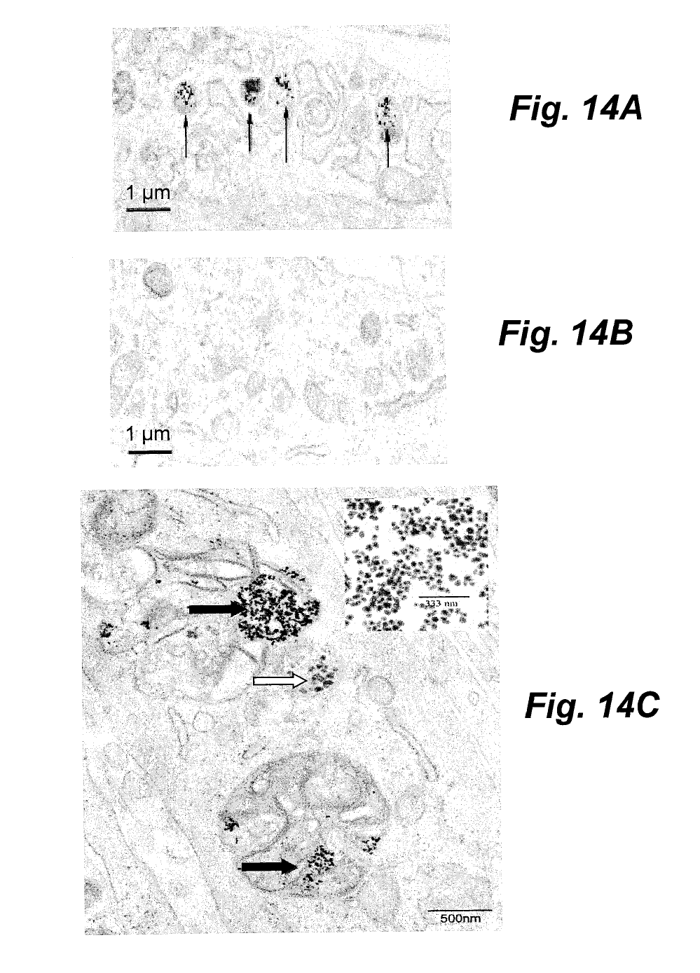

To obtain higher resolution images of intracellular nanoparticle distribution, a variant of NP1 containing a cobalt ferrite metal core (NP3, Table 1) was used to enable nanoparticle visualization by Transmission Electron Microscopy (TEM). As shown in FIGS. 14A-14C, NP3 was associated with organelles in those MC3T3 cells treated with nanoparticles (FIG. 14A), but not in untreated cells (FIG. 14B). Nanoparticles were not observed in rough or smooth endoplasmic reticulum or in mitochondria. Higher magnification images are shown in FIG. 14C. Two sizes of electron dense particles were observed, a small particle likely corresponding to uncoated metal cores (arrows) and a larger particle probably corresponding to 50 nm silica coated cores (white arrow). Monodispersed NP3 in solution is shown for comparison in the FIG. 14C, inset. The data show that silica nanoparticles are internalized through endosomal transport, and remain within certain cellular structures for a period of time prior to the silica shell being degraded within specific cellular organelles.

### NP1 Internalization Rate is Cell Type Dependent.

To assess the capacity of NP1 to internalize and affect the function of different non-bone-lineage related cell types the uptake of NP1 in HEK293 human embryonic kidney cells, JB6 CL41 mouse epidermal cells and NIH3T3 mouse fibroblasts, was compared to that with RAW264.7 and MC3T3 cells. When exposed to a 50 μg/ml dose of nanoparticle for 18 hr, NP1 fluorescence was strongly detected in RAW267.4 and MC3T3 cells, while the fluorescent intensity was dramatically lower in the non-bone-lineage cells examined. High camera exposure settings did show that nanoparticles were present in all cell types. These data indicate an increased propensity for NP1 internalization into certain cell types, rather than in others.

### NP1 Stimulates Bone Formation and Suppresses Bone Resorption by Inhibiting Ff-κB Signal Transduction.

There was similarity between NP1 action and that of TNFα on osteoclasts and osteoblasts. Thus, TNFα is a potent stimulator of the NF-κB signal transduction pathway which is critical to osteoclast differentiation. Consequently TNFα has the capacity to synergize with, and amplify, RANKL-induced osteoclastogenesis, as reported by Cenci et al., J. Clin Invest 106: 1229-1237 (2000), and Lam et al., J Clin Invest 106: 1481-1488 (2000), incorporated herein by reference in their entireties. In contrast to osteoclasts, TNFα-induced NF-κB activity is potently inhibitory of osteoblast differentiation in vitro and of bone formation in vivo (Li et al., J. Bone Miner. Res. 22: 646-655 (2007), incorporated herein by reference in its entirety). Also, NF-κB suppression inhibits ovariectomy—induced osteoclastic resorption in vivo (Strait et al., Int. J. Mol. Med. 21: 521-525 (2008), incorporated herein by reference in its entirety) and stimulates osteoblastic differentiation and mineralization in vitro. While not wishing to be bound by any one theory, these data have indicated that NP1 may accomplish its effects on bone cells by modulating NF-κB levels or activity. Accordingly, MC3T3 and RAW264.7 cells were transfected with a luciferase reporter specifically driven by three tandem NF-κB consensus motifs.

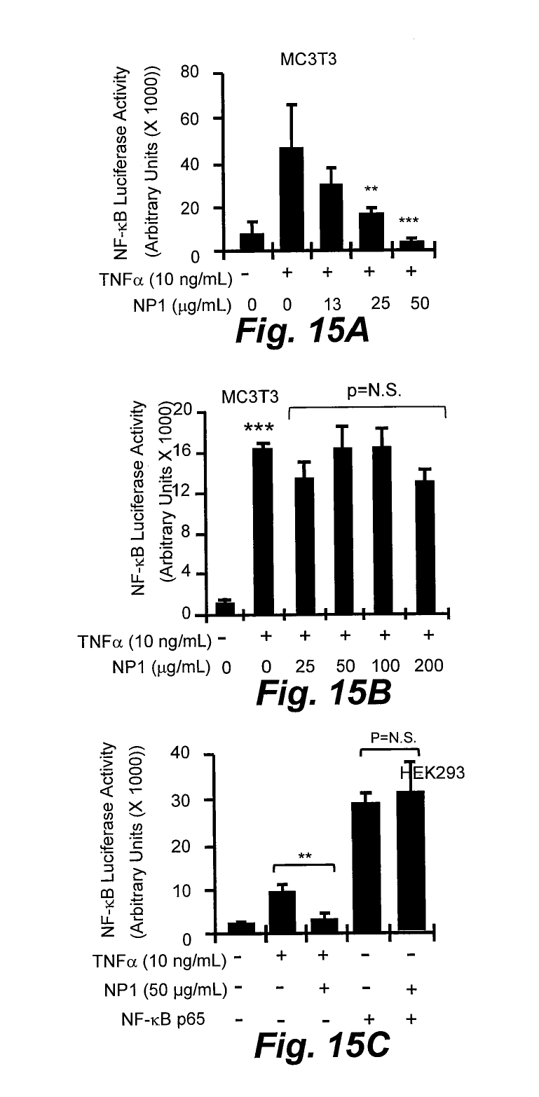

Cells were stimulated with TNFα to induce NF-κB activity and treated with NP1. NP1 dose-dependently suppressed TNFα-induced NF-κB activity in MC3T3, as shown in FIG. 15A. NP1 did not suppress TNFα-induced NF-κB reporter activity in HEK293 cells (FIG. 15B), and therefore NP1 action on NF-κB is not universal and is limited to certain types of cells.

To investigate whether NP1 sequesters NF-κB or prevents its activation, the NF-κB subunit p65 was over-expressed in MC3T3 cells. p65 potently induced NF-κB reporter transcription, but was unaffected by high concentrations of NP1 (as shown in FIG. 15C). Thus, NP1 suppresses NF-κB activation rather than directly associating with and impeding the nuclear translocation of free NF-κB subunits.

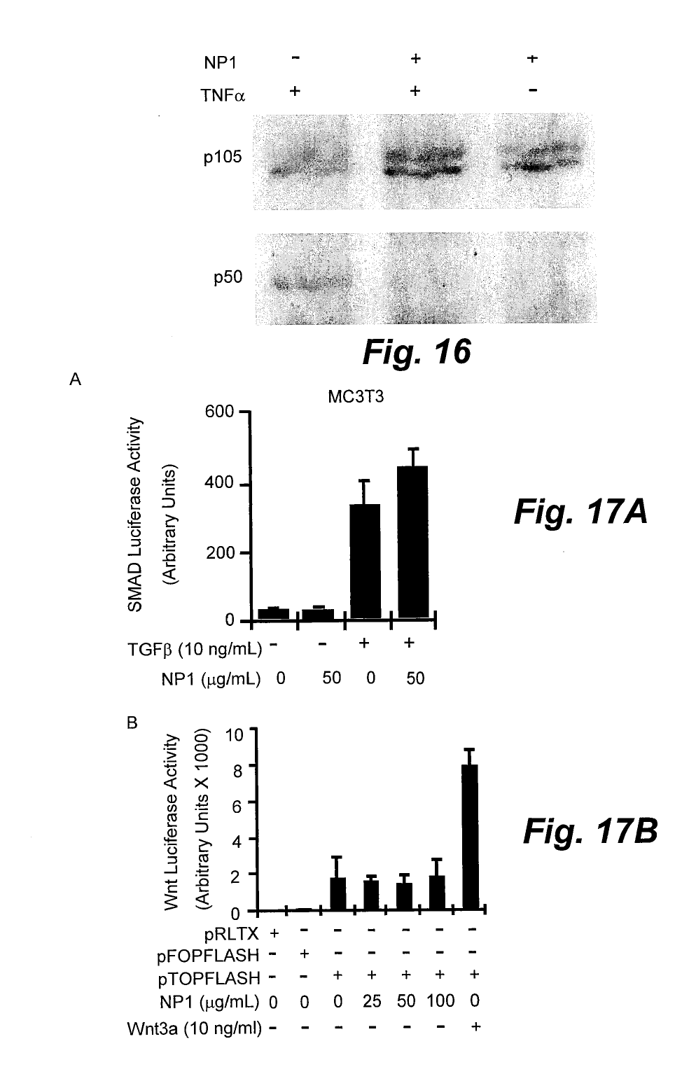

Three major NF-κB subunits, p50, p52 and p65, have been implicated in NF-κB signaling in osteoblasts and osteoclasts (Abu-Amer, J. Clin. Invest. 107: 1375-1385 (2001); Nanes, Gene 321: 1-15 (2003)). p50 is generated from a precursor, p105, which comprises the NF-κB subunit fused to an inhibitory IκB domain. The active p50 subunit is released from the precursor peptide by proteolytic processing in the proteasome. As the data shown in FIG. 15C suggested that NP1 acts by preventing the activation of NF-κB rather than sequestering it like an IκB, the effect of NP1 on proteolytic cleavage of p105 into the p50 NF-κB subunit was examined. MC3T3 cells were treated with NP1, in the presence or absence of TNFα, a potent stimulator of p105 cleavage to p50. The data showed that NP1 suppresses the TNFα-driven conversion of the p105 precursor into p50 (FIG. 16), thus reducing the concentrations of this NF-κB subunit available for heterodimerization and nuclear translocation.

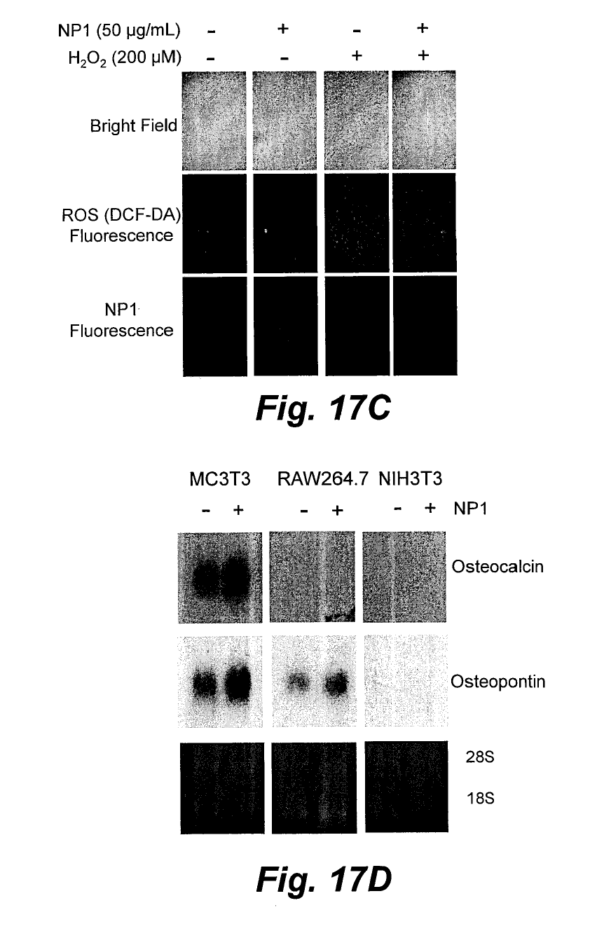

To assess the specificity of NP1 action on NF-κB, the effect of NP1 on other common signal transduction pathways known to be involved in osteoblast and/or osteoclast differentiation were examined. TGFβ and BMPs are potent commitment and differentiation factors, respectively for osteoblast differentiation (Janssens et al., Bone Endocrin. Rev. 26: 743-774 (2005), incorporated herein by reference in its entirety). TGFβ is also reported to augment osteoclastogenesis in vitro (Quinn et al., J. Bone Miner. Res. 16, 1787-1794 (2001), incorporated herein by reference in its entirety). TGFβ and BMPs signal predominantly through SMADS, consequently, the effect of NP1 on SMAD signal transduction was tested by transfecting MC3T3 cells with a luciferase reporter driven by 3 tandem SMAD4 binding sites, a motif recognized and transactivated by all SMAD heterodiamers. However, NP1 had no effect on the capacity of TGFβ to transactivate this reporter. Likewise, the Wnt pathway is another potent inducer of osteoblast formation. To evaluate the effect of NP1 on Wnt signaling, MC3T3 cells were transfected with the βCatenin responsive TCF-reporter construct pTOPFLASH, or its inactive control pFOPFLASH. While recombinant Wnt3a potently induced Wnt activity, NP1 showed no capacity to stimulate Wnt expression, as shown in FIG. 17B.

Reactive oxygen species (ROS) are potent stimulators of osteoclastogenesis (Grassi et al., Proc. Natl. Acad. Sci. U.S.A. 104: 15087-15092 (2007)) and have been associated with osteoblast and osteocyte apoptosis (Almeida et al., J. Biol. Chem. 282, 27285-27297 (2007)). Certain nanoparticle formulations are known to possess ROS scavenging activities. To investigate the potential for NP1 to scavenge ROS, MC3T3 cells were treated with NP1 for 1 hr, then cells were loaded with 2′,7′-dichlorofluorescein diacetate (DCF-DA), a commonly used probe to detect the formation of ROS in cells in culture. Chemically reduced and acetylated forms of DCF-DA are non-fluorescent until the acetate groups are removed by intracellular esterases and oxidation occurs in the cell. Oxidation of DCF-DA in MC3T3 cells was monitored by fluorescent microscopy, using H₂O₂ to stimulate ROS production. NP1 neither generated ROS nor sequestered ROS (FIG. 17C). Identical results were obtained 24 hours after addition of NP1.

The specificity of NP1 action on basal osteocalcin and osteopontin induction was examined using Northern blots. As shown in FIG. 17D, NP1 upregulated basal osteocalcin and osteopontin in MC3T3 cells, but failed to significantly induce these genes in the non-osteoblastic RAW264.7 and NIH3T3 cells. While not wishing to be bound by any one theory, NP1 appears to induce osteoblastic gene products through the stimulation of differentiation of preosteoblasts along the osteoblast lineage, rather than by direct activity on osteoblastic gene promoters, and NP1 likely achieves its stimulatory activity on osteoblastogenesis and its inhibitory activity on osteoclastogenesis predominantly, if not exclusively, through suppression of NF-κB signal transduction.

Nanoparticle NP1 Binds to Bone Surfaces Through Association with Hydroxyapatite.

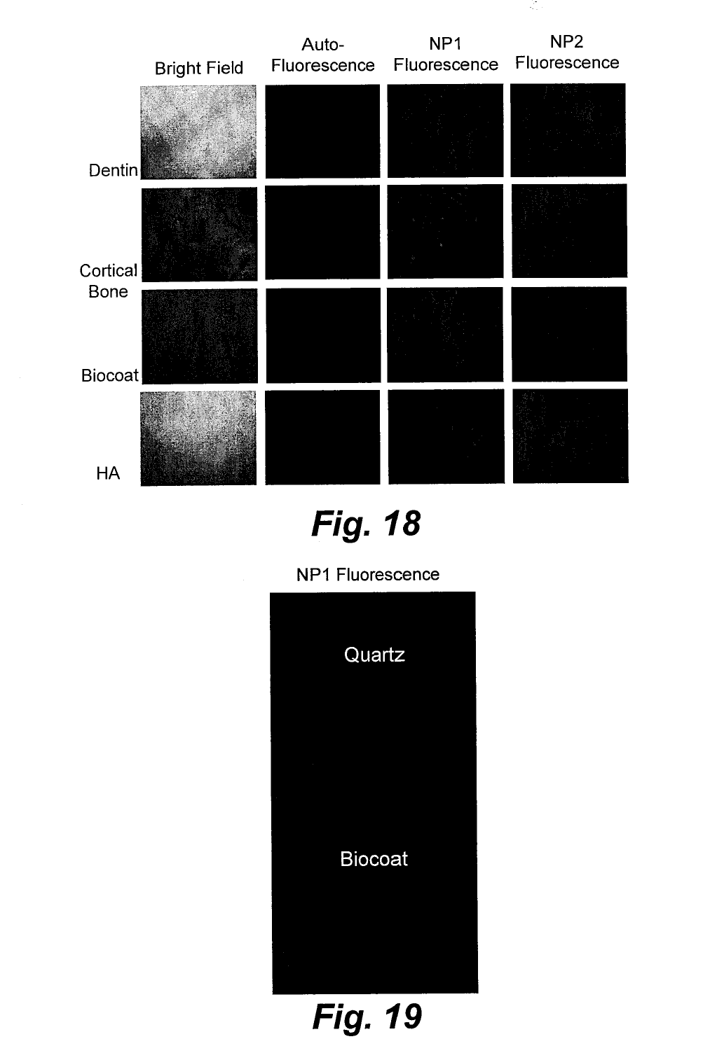

In vitro examination of NP1 fluorescence in mineralized nodules indicated that these nanoparticles may not only be retained in cells, but may physically associate with mineral. Accordingly, dentine slices and devitalized bovine cortical bone chips were incubated with NP1 or NP2 for 2 hr, washed extensively, and examined by fluorescence microscopy. Both NP1 and NP2 were found to bind strongly to the dentine and cortical bone surfaces as well as to OSTEOLOGIC BIOCOAT™, a calcium phosphate film immobilized to a quartz substrate that mimics hydroxyapatite and is resorbable by osteoclasts (FIG. 18). NP1 and NP2 bound strongly to the calcium phosphate, but not to the quartz substrate (FIG. 19).

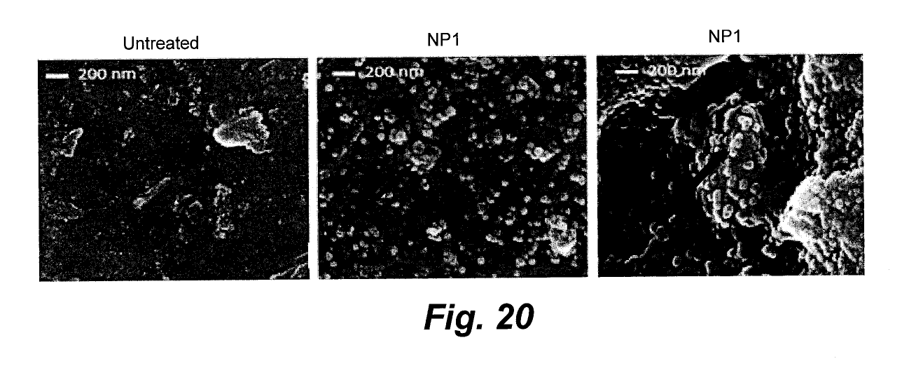

To directly visualize nanoparticle adhesion to dentine, NP1 was further incubated with dentine slices and examined by Scanning Electron Microscopy (SEM). Nanoparticles were observed to coat the dentine surface (red arrow) and to also penetrate and coat pits in the dentine surface (FIG. 20). Attesting to specificity, QD633, a commercial quantum dot nanoparticle failed to bind to the dentine surface.

### NP4 Enhances BMD and Indices of Bone Structure In Vivo.

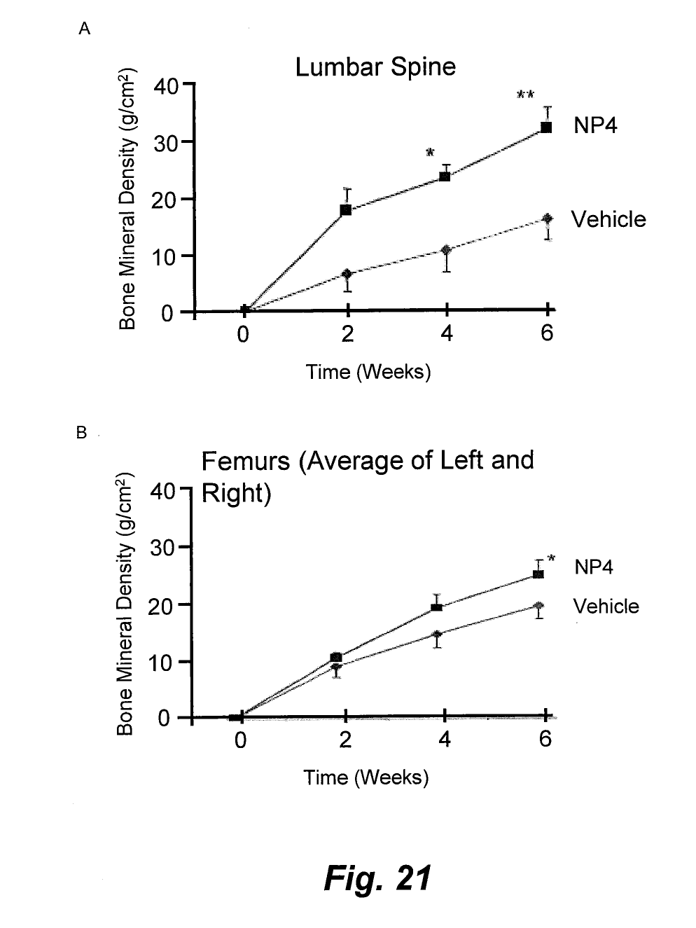

NP1 and NP2 stimulate bone formation and suppress bone resorption in vitro, as shown by the results illustrated in FIGS. 1-10. To determine whether nanoparticles can augment bone mass in vivo, NP4, a PEGylated derivative of NP2 suitable for use in vivo, was injected into mice once per week for 5 weeks. BMD was followed prospectively every two weeks for 6 weeks using DXA. NP4 administration in vivo led to a significant 10% increase in BMD at the lumbar spine by 4 weeks and by 15% at 6 weeks, as shown in FIG. 21A. BMD in the femurs increased at a slower rate achieving a more modest 5% increase by 6 weeks of treatment (FIG. 21B).

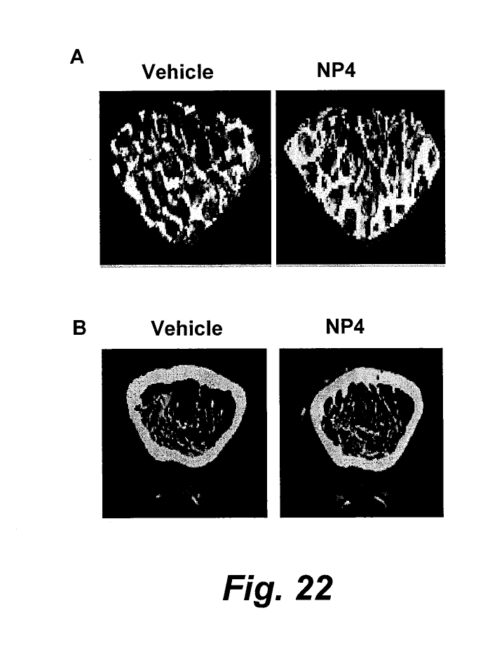

Lumbar spine and femoral trabecular bone compartments were further evaluated by micro-CT. Representative three dimensional reconstructions of the spine, as illustrated in FIG. 22A, and the femur (FIG. 22B) from untreated and NP4 treated mice, clearly showed increased trabecular bone mass in NP4 treated mice.

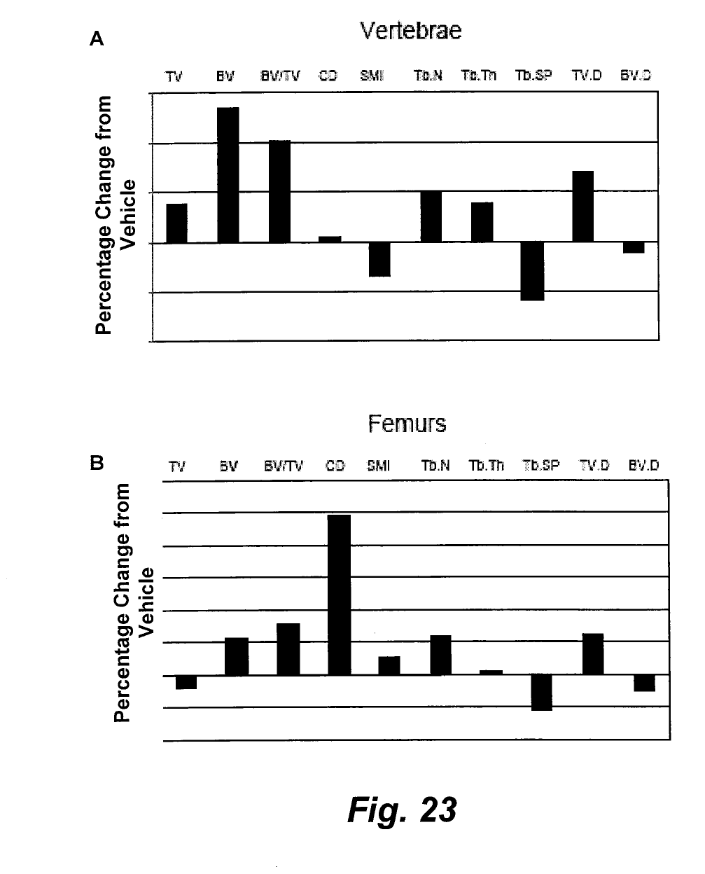

As shown in FIG. 23A (vertebrae) and FIG. 23B (femur), trabecular structural indices indicated an increase in the ratio of trabecular bone volume (BV) to tissue volume (TV), in NP4-treated mice, a consequence of increased bone volume. TV was unchanged. Elevated BV was consistent with enhanced Trabecular Thickness (Tb. Th.), Trabecular Number (Tb. N.), and Trabecular Connection Density (Conn. D.), with a corresponding decrease in Trabecular Spacing (Tb. Sp.). BMD, as a function of TV (TV. D), was somewhat elevated, indicative of an increase in bone mass.

The accumulated data show that silica-derived nanoparticle formulations of the disclosure may stimulate osteoblast differentiation and mineralization and suppress osteoclast formation in vitro and increase bone mass in vivo. These nanoparticles probably act by suppressing the NF-κB transcription factor.

The data support the hypothesis that NP4 increases bone mass by both stimulating bone formation in vivo. Additionally, there may be a third mechanism that contributes to the total increase in bone mass by a direct and passive increase in bone mass through association of NP4 with hydroxyapatite coating the trabecular surfaces and directly increasing their volume and BMD. The capacity of these nanoparticles to bind hydroxyapatite may lead to enhanced skeletal sequestration and preferential retention in the bone compartment leading to elevated local concentrations in the bone microenvironment. This, along with the preferential internalization of NP1 and NP2 into certain cell types such as osteoclast- and osteoblast-lineage cells, lowers the potential for toxicity in vivo, and reduce the potential for targeting non-bone related tissues.

NP1 not suppressing NF-κB activity in HEK293 cells is most likely a consequence of the reduced endocytosis observed for NP1 into this cell line. Consequently, the intracellular concentration of NP1 would not reach a level capable of suppressing the NF-κB pathway. This contrasts with RAW264.7 cells and MC3T3 cells which rapidly internalized high concentration of nanoparticles.

NP1 administration in vivo leads to a significant increase in BMD at the lumbar spine within 6 weeks of treatment. A more modest increase was seen at the femur over the same period of time. As the spine contains high concentrations of trabecular bone relative to the femurs, which are more cortical rich, NP4 appears to have a more pronounced effect on trabecular bone accrual than on cortical bone.

Mesoporous silica nanoparticles has been reported to have no effect on viability, proliferation, immunophenotype, or differentiation of mesenchymal stem cells (osteoblast precursors) in vitro (Huang et al., 2005). In contrast, the data accrued from nanoparticles NP1 and NP2 of the present disclosure reveal potent osteoblastogenic activity of silica nanoparticles. NP1 and NP2 are each about 50 nm in size, compared to the 110 nm nanoparticles of earlier, negative, studies. Additionally, NP1 and NP2 have distinctly rounded shapes, whereas the earlier negative nanoparticles (Huang et al, FASEB J. 19: 2014-2016 (2005)) had a hexagonal conformation.

The data now show that certain silica-derived nanoparticles have the capacity to bind to hydroxyapatite and stimulate bone formation while simultaneously repressing bone resorption, in vitro and in vivo. While not wishing to be bound by any one theory, these activities are likely a consequence of a natural propensity of these nanoparticles to inhibit NF-κB in osteoclasts and osteoblasts. These nanoparticles, therefore, may have significant potential for use as novel dual-anabolic and anti-catabolic pharmaceuticals for amelioration of bone disease and in fracture prevention and healing.

One aspect of the disclosure, therefore, encompasses methods of modulating the formation of a population of osteoblasts, comprising contacting a cell with an effective amount of a composition comprising a silica-based nanoparticle, wherein the silica-based nanoparticle modulates the formation of a population of osteoblasts.

In embodiments of this aspect of the disclosure, the cell may be selected from the group consisting of: an isolated stem cell, a stem cell in an animal or human subject, an isolated osteoblast progenitor cell, an osteoblast progenitor cell in an animal or human subject, an isolated osteoblast, an osteoblast in an animal or human subject, or a combination thereof.

In some embodiments of this aspect of the disclosure, the population of osteoblasts increases.

In some embodiments of the disclosure the silica-based nanoparticle may comprise a metallic core and a silicaceous shell disposed on the metallic core.

In other embodiments of the disclosure, the nanoparticles may further comprise a polymeric protective coat. In some of these embodiments, the protective coat may be comprised of polyethylene glycol, polyvinyl pyrrolidone, PTMA, or PMP, or any combination thereof.

In another embodiment, the silica-based nanoparticle of the disclosure may further comprise a label. In this embodiment, the label may be a fluorescent label.

Another aspect of the disclosure encompasses methods of promoting bone formation, the embodiments comprising delivering to a subject in need thereof, an effective dose of a pharmaceutically acceptable composition comprising a silica-based nanoparticle, wherein the silica-based nanoparticle increases the formation of osseous material in the subject.

In embodiments of this aspect of the disclosure, the pharmaceutically acceptable composition may further comprise a carrier.

In embodiments of the disclosure, the silica-based nanoparticle may increase the proliferation of a population of osteoblasts in the subject, thereby promoting the formation of osseous material.

In embodiments of the disclosure the silica-based nanoparticle may decrease the loss of osseous material from a bone of the subject. In this embodiment of the disclosure, the silica-based nanoparticle may decrease the loss of osseous material from a bone of the subject by inhibiting the activity of osteoclasts in the subject.

In this aspect of the disclosure, embodiments of the method may comprise the silica-based nanoparticle inhibiting osteoclast activity in the subject by inhibiting an increase in a population of osteoclasts, inhibiting the differentiation of cells of a population of monocyte-macrophage cells into preosteoclasts, fusion of preosteoclasts into osteoclasts, or a combination thereof.

In other embodiments of the disclosure, the silica-based nanoparticle may decrease the loss of osseous material from a bone of the subject by inhibiting the ability of osteoclasts to remove osseous material from a bone of the subject.

In yet other embodiments of the disclosure, the silica-based nanoparticle adheres to a mineral component of the osseous material of the subject, thereby increasing the volume of osseous material.

In the embodiments of this aspect of the disclosure, the silica-based nanoparticle may comprise a metallic core and a silicaceous shell disposed on the metallic core.

In other embodiments of this aspect of the disclosure, the nanoparticles may further comprise a polymeric protective coat. In some of these embodiments, the protective coat may be comprised of polyethylene glycol, polyvinyl pyrrolidone, PTMA, PMP, or a combination thereof.

In other embodiments, the silica-based nanoparticle further comprises a label. wherein the label may be a fluorescent label.

Still another aspect of the invention encompasses embodiments of pharmaceutically acceptable compositions comprising an effective dose of a silica-based nanoparticle, wherein the silica-based nanoparticle can increase the formation of osseous material in the subject.

Embodiments of this aspect of the disclosure may further comprise a carrier.

In embodiments of the disclosure, the silica-based nanoparticle may increase the proliferation of a population of osteoblasts in the subject, thereby increasing the formation of osseous material.

In other embodiments of the disclosure, the silica-based nanoparticle may decrease the loss of osseous material from a bone of the subject. In these embodiments, the silica-based nanoparticle may decrease the loss of osseous material from a bone of the subject by inhibiting the activity of osteoclasts in the subject.

In yet other embodiments of the disclosure, the silica-based nanoparticle may inhibit osteoclast activity in the subject by inhibiting osteoclast activity in the subject by inhibiting an increase in a population of osteoclasts, inhibiting the differentiation of cells of a population of monocyte-macrophage cells into preosteoclasts, fusion of preosteoclasts into osteoclasts, or a combination thereof.

In still other embodiments of the disclosure, the silica-based nanoparticle may decrease the loss of osseous material from a bone of the subject by inhibiting the ability of osteoclasts to remove osseous material from a bone of the subject.

In other embodiments of this aspect of the disclosure, the silica-based nanoparticle of the compositions adheres to a mineral component of the osseous material of the subject, thereby increasing the volume of osseous material.

In the embodiments of this aspect of the disclosure, the silica-based nanoparticle may comprise a metallic core and a silicaceous shell disposed on the metallic core.

In other embodiments of this aspect of the disclosure, the nanoparticles may further comprise a polymeric protective coat. In some of these embodiments, the protective coat may be comprised of polyethylene glycol, polyvinyl pyrrolidone, PTMA, or PMP, or any combination thereof

In embodiments of the disclosure, the silica-based nanoparticle may further comprise a label, where the label may be a fluorescent label.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include±1%, ±2%, ±3%, ±4%, ±5%, ±6%, ±7%, ±8%, ±9%, or ±10%, or more of the numerical value(s) being modified.

### EXAMPLES

### Example 1

### Synthesis of Silica-Based Nanoparticles

An exemplary protocol, slightly modified from Yoon et al., Angew. Chem. Int. Ed. 44, 1068-1071 (2005) (incorporated herein by reference in its entirety) is as follows:

FeCl₃.6H₂O, CoCl₂.6H₂O, and Fe(NO₃)₃.9H₂O, allyl iodide, rhodamine B, cesium carbonate, trimethoxysilane, and platinum on activated charcoal (Pt/C) were purchased from Aldrich and tetraethyl orthosilicate was purchased from TCI. N-trimethoxysilylpropryl-N,N,N-trimethylammonium chloride, (MeO)₃Si—PTMA, and Methyl(polyethyleneoxy)-propryl-trimethoxy-silane, (MeO)₃Si—PEG, were purchased from Gelest, and all the reagents were used without further purification.

### Synthesis of (Trimethoxysilylpropyl)-Rhodamine B; TMSP-RhB

This synthesis scheme is illustrated in FIG. 25. 0.5 g (1.04 mmol) of rhodamine B, 0.54 g (3.12 mmol) of allyl iodide, and 1.02 g (3.12 mmol) of cesium carbonate were dissolved in dried DMF, and was stirred at 60° C. for a day. The mixture was extracted with methylene chloride and washed with water 3 times. Organic layer was concentrated by rotary evaporator. The residual DMF was removed by vacuum distillation. Finally, 0.511 g (94.6%) of dark and reddish powder was taken by separating column chromatography with methylene chloride and methanol. 50 mg (0.096 mmol) of the obtained allyl-rhodamine B, 25 mg (0.192 mmol) of trimethoxysilane, and catalytic amounts of Pt/C was dissolved in freshly dried methanol. After reflux for 1 day, catalyst was removed by Celite filtration. Solvent and excess trimethoxysilane were removed in vacuum. 60.8 mg (98.7%) of TMSP-RhB was obtained as dark-reddish oil.

Allyl-rhodamine B; ¹H NMR (CDCl₃); δ ppm 8.31 (d, 1H), 7.82 (tt, 2H), 7.34 (d, 1H), 7.10 (d, 2H), 6.93 (dd, 2H), 6.80 (d, 2H), 5.70 (m, 1H), 5.20 (dd, 2H), 4.53 (d, 2H), 3.68 (q, 8H), 1.34 (t, 12H) ¹³C NMR (CDCl₃); δ ppm 164.61, 158.62, 157.65, 155.45, 133.48, 133.20, 131.28, 131.19, 131.04, 130.41, 130.18, 129.81, 119.04, 114.30, 113.43, 96.21, 66.02, 46.23, 12.70 MS (FAB+) m/z 483

### General Method for Synthesis of Fluorescent Silica Nanoparticles

SiO₂(RhB): 20 mg of trimethoxysilyl-propyl-rhodamine B (TMSP-RhB) and 0.86 g of tetraethyl orthosilicate was dissolved in ethanol. 1 ml of ammonia and water were added, kept stirring. After 4 hours, fluorescent silica nanoparaticles were taken from centrifugation, and the supernatant was removed. The precipitate was dispersed in ethanol. This washing process was repeated 3 times. Finally, nanoparticles were dispersed in ethanol for the surface-modified step or water for the cell culture. Their size and shape were characterized by TEM and SEM. Table 2 summarizes the conditions of synthesizing silica nanoparticles with various sizes.

TABLE 2
Conditions for size-controlled SiO₂(RhB) nanoparticles

| | Molar concentration (M) | | | |
|---|---|---|---|---|
| Entry | TEOS | Ammonia | Water | Size (nm)* |
| 1 | 0.082 | 0.178 | 2.00 | 30 |
| 2 | 0.115 | 0.178 | 2.00 | 50 |
| 3 | 0.123 | 0.533 | 0.89 | 100 |

*The size of silica nanoparticles was measured by TEM and SEM.

MNP-SiO₂(RhB): Cobalt ferrite solution (34.7 ml, 20 mg MNP ml⁻1 solution in water) was added to polyvinylpyrrolidone solution (PVP; 0.65 ml; Mr 55,000 Da, 25.6 gL⁻1 in H₂O), and the mixture was stirred for 1 day at room temperature. The PVP-stabilized cobalt ferrite nanoparticles were separated by addition of aqueous acetone (H₂O/acetone=1/10, v/v) and centrifugation at 4000 rpm for 10 min. The supernatant solution was removed, and the precipitated particles were redispersed in ethanol (10 ml). Multigram-scale preparation of PVP-stabilized cobalt ferrite nanoparticles was easily reproduced in this modified synthetic method. A mixed solution of TEOS and (trimethoxysilylpropyl)-rhodamine B (TEOS/TMSP-RhB molar ratio=0.3/0.04) was injected into the ethanol solution of PVP-stabilized cobalt ferrite. Polymerization initiated by adding ammonia solution (0.86 ml; 30 wt % by NH₃) as a catalyst produced cobalt ferrite—silica core-shell nanoparticles containing organic dye. These nanoparticles were dispersed in ethanol and precipitated by ultra-centrifugation (18000 rpm, 30 min). This washing process was repeated 3 times, and nanoparticles were finally dispersed in ethanol for the surface-modified step or in water for the cell culture.

45 mg of purified SiO₂(RhB) or core-shell nanoparticles, MNP-SiO₂(RhB), were redispersed in absolute ethanol (10 ml) and then treated with 2-[methoxy(polyethyleneoxy)propyl]trimethoxysilane (PEG-Si(OMe)₃; 125 mg, 0.02 mmol), CH₃O(CH₂CH₂O)₆₋₉—CH₂CH₂CH₂Si(OCH₃)₃, at pH 12 (adjusted with NH₄OH). The resulting SiO₂(RhB)-PEG or MNP-SiO₂(RhB)-PEG was washed and centrifuged in EtOH several times and characterized by IR spectroscopy (C—H stretching band at 2800-2900 cm⁻¹). All the prepared nanoparticles were characterized by TEM, FT-IR, UV/V is absorption and emission spectroscopy, and vibrating sample magnetometer (VSM) measurements.

### Example 2

### Animals

Female C57BL6 mice 6 weeks of age were purchased from Jackson Laboratories. Mice were acclimated for 2 weeks in the animal facility before use. Mice were housed in sterile polycarbonate cages with corn cob bedding on static racks and given gamma-irradiated 5V02 phytoestrogen-free mouse chow (Purina Mills, St. Louis, Mo.), and autoclaved water ad libitum. The animal facility was kept at 23°±1° C., with 50% relative humidity and a 12/12 light/dark cycle.

### Example 3

### Micro-Computed Tomography (Micro-CT)

Micro-CT was performed using a μCT40 scanner, (Scanco Medical, Bassersdorf, Switzerland) as previously described (Li et al., Blood 109: 3839-3848 (2007), incorporated herein by reference in its entirety). Briefly, after careful dissection of muscle tissue, the right femur was fixed in 10% neutral buffered formalin for 48 hr and stored in 70% ethanol at 4° C. until analysis. Micro-CT analysis was performed by an operator blinded to the nature of the specimens. Bones were scanned at a resolution of 12 μm. For each sample, 50 slices were taken at the identical starting position and covering a total area of 600 μm proximal to the distal metaphyses. Static trabecular measurements were made using a cylindrical core sample that excluded cortical bone, with contouring for all subsequent slices. For visual representation one representative sample from each group was randomly selected for detailed three-dimensional (3D) reconstruction of core images from individual micro-CT slices.

### Example 4

### Biochemical Indices of Bone Formation and Resorption

Osteocalcin, a sensitive biochemical marker of in vivo bone formation was measured in mouse serum in vehicle or NP4 injected mice following an overnight fast, using a rodent specific ELISA, Rat-Mid (Immunodiagnostic Systems Inc. Fountain Hills, Ariz.).

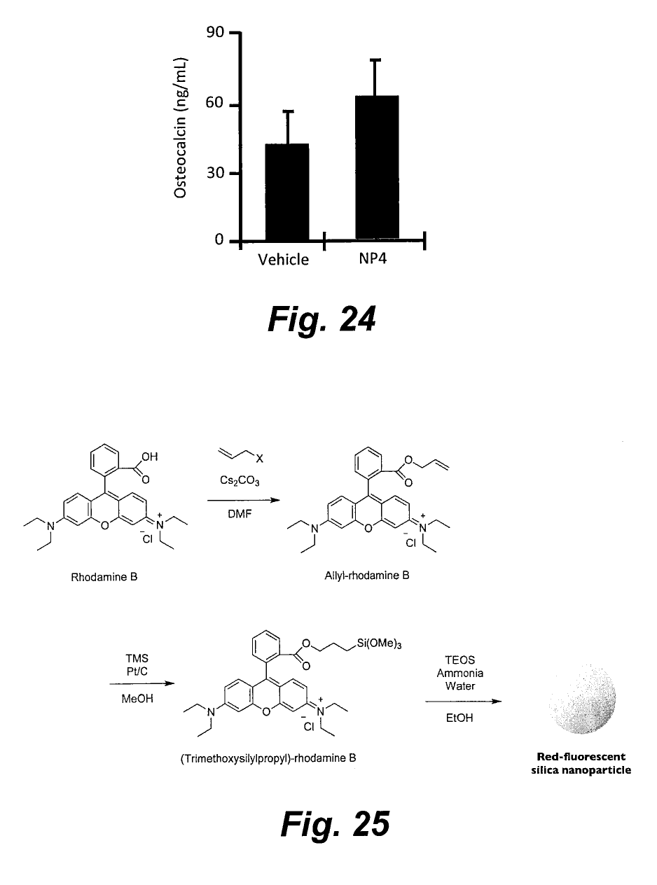

The data, as shown in FIG. 24, shows a percentage increase in indices of bone formation of 50.0%. Data represent average±SEM of n=9 Vehicle and 8 NP4 injected mice. These data support the contention that silica based nanoparticles have the capacity to stimulate bone formation in vivo.

### Example 5

### Quantitation of Bone Mineral Density

In vivo BMD measurements of total body, lumbar spine (spine), and femurs (left and right femurs were averaged for each mouse) were made by Dual-energy X-ray Absorptiometry (DXA) using a PIXImus2 bone densitometer (GE Medical Systems) as previously described (Toraldo et al., Proc. Natl. Acad. Sci. U.S.A. 100, 125-130 (2003) and incorporated herein by reference). The intra-assay variability of this technique was <0.9%.

### Example 6

### NF-κB, Wnt and Smad Reporter Constructs and Luciferase Assays

MC3T3 and RAW264.7 cells were transfected with the NF-κB responsive reporter pNFκB-LUC (BD Biosciences), the Smad reporter pGL3-Smad (Li et al., 2007a) or the Wnt-responsive reporter TOPFLASH or its negative control FOPFLASH (Invitrogen (Carlsbad, Calif.), using Lipofectamine 2000 (Invitrogen). In some experiments, cells were co-transfected with a p65 expression vector (Lu et al., J. Cell Biochem. 92, 833-848 (2004), incorporated herein by reference in its entirety). In these experiments total plasmid concentration was equalized using pBluescript KS⁺ (Invitrogen). Transfection efficiency was verified in replicate cultures using pRL-SV40. The presence of nanoparticles in culture was confirmed to have no effect on transfection efficiency. Luciferase activity was measured on a microplate luminometer (Turner Designs, Sunnyvale, Calif.) using the luciferase assay system of Promega Corporation with passive lysis buffer.

### Example 7

### Osteoclast Formation

Osteoclasts were cultured in 96 well plates using the monocytic cell line RAW264.7 (1×10⁴ cells per well) or from immunomagnetically purified CD11b monocytes (1×10⁵ cells per well) purified from mouse spleens. Osteoclast development was stimulated by addition of RANKL (25 ng/ml) in the presence of 2.5 μg/ml of a cross-linking antibody (mouse anti-6×-histidine). Primary monocytes additionally received 25 ng/ml of Macrophage Colony Stimulating Factor (M-CSF). Cultures were treated with NP1 or NP2 as indicated. After 7-10 days of culture, cells were stained for Tartrate Resistant Acid Phosphatase (TRAP) and the number of mature osteoclasts (TRAP positive and 3 nuclei) were counted under light microscopy and normalized for size on the basis of every 3 nuclei representing one osteoclast. Each data point was performed in quadruplicate and averaged for each experiment.

### Example 8

### MC3T3 and Primary Stromal Cell Mineralization Assays

The clonal osteoblastic cell line, MC3T3-E1, clone 14, was from the American Type Culture Collection and has been characterized in detail (Wang et al., J. Bone Miner. Res 14: 893-903 (1999)), incorporated herein by reference in its entirety). Primary stromal cells were isolated as previously described (Gao et al., Cell Metab. 8: 132-145 (2008)), incorporated herein by reference in its entirety). MC3T3 cells or primary stromal cells were plated at confluence in 96-well plates and differentiated to osteoblasts in αMEM supplemented with 10% FBS, 50 μg L-ascorbate and, 10 mM β-glycerophosphate as previously described (Li et al., J. Bone Miner. Res. 22: 646-655 (2007), incorporated herein by reference in its entirety). Mineralization was visualized by fixing cells in 75% ethanol for 30 minutes at 4° C. followed by staining with alizarin red-S (40 mM) for 10 mins. Excess stain was removed by copious washing with distilled water.

### Example 9

### Nanoparticle Association with Bone Surfaces

Dentin slices (Immunodiagnostic Systems Inc.), devitalized cortical bovine bone chips, BD Biocoat Osteologic discs (BD Biosciences), and hydroxyapatite (Sigma) were incubated for 2 hr in 96 well plates with NP1 or NP2 (50 μL) of stock (1.2 mg/ml). Nanoparticles were removed and wells washed vigorously 3 times for 15 mins with 100 μL of PBS with vigorous shaking. Bone and bone surrogates were photographed under light or fluorescence microscope.

### Example 10

### Statistical Analysis

Statistical significance was determined using GraphPad InStat version 3 for Windows XP (GraphPad Software). Simple comparisons were made using unpaired 2 tailed Students t test. Multiple comparisons were performed by One-way ANOVA or Repeated Measures ANOVA with Tukey-Kramer post test. p≦0.05 was considered statistically significant. Groups were confirmed to be normally distributed using the Kolmogorov-Smirnov test. All data is presented as mean±S.D. p.≦0.05 was considered significant. (*) p<0.05; (**) p<0.01; (***) p<0.001; N.S.=not significant).

We claim:

1. A method of modulating the formation of a population of osteoblasts, comprising contacting a cell with an effective amount of a composition comprising a silica-based nanoparticle, wherein the silica-based nanoparticle modulates the formation of a population of osteoblasts.

2. The method of claim 1, wherein the cell is selected from the group consisting of: an isolated stem cell, a stem cell in an animal or human subject, an isolated osteoblast progenitor cell, an osteoblast progenitor cell in an animal or human subject, an isolated osteoblast, an osteoblast in an animal or human subject, or a combination thereof.

3. The method of claim 1, wherein the population of osteoblasts increases.

4. The method of claim 1, wherein the silica-based nanoparticle comprises a metallic core and a silicaceous shell disposed on the metallic core.

5. The method of claim 1, wherein the silica-based nanoparticle further comprises a polymeric protective coat.

6. The method of claim 5, wherein the protective coat is comprised of polyethylene glycol, polyvinyl pyrrolidone, PTMA, or PMP, or any combination thereof.

7. The method of claim 1, wherein the silica-based nanoparticle further comprises a label.

8. The method of claim 7, wherein the label is a fluorescent label.

9. A method of promoting bone formation, comprising delivering to a subject in need thereof, an effective dose of a pharmaceutically acceptable composition comprising a silica-based nanoparticle, wherein the silica-based nanoparticle increases the formation of osseous material in the subject.

10. The method of claim 9, wherein the pharmaceutically acceptable composition further comprises a carrier.

11. The method of claim 9, wherein the silica-based nanoparticle increases the proliferation of a population of osteoblasts in the subject, thereby promoting the formation of osseous material.

12. The method of claim 9, wherein the silica-based nanoparticle decreases the loss of osseous material from a bone of the subject.

13. The method of claim 12, wherein the silica-based nanoparticle decreases the loss of osseous material from a bone of the subject by inhibiting the activity of osteoclasts in the subject.

14. The method of claim 13, wherein the silica-based nanoparticle inhibits osteoclast activity in the subject by inhibiting an increase in a population of osteoclasts; inhibiting the differentiation of cells of a population of monocyte-macrophage cells into preosteoclasts; inhibiting the fusion of preosteoclasts into osteoclasts; or a combination thereof.

15. The method of claim 11, wherein the silica-based nanoparticle decreases the loss of osseous material from a bone of the subject by inhibiting the ability of osteoclasts to remove osseous material from a bone of the subject.

16. The method of claim 9, wherein the silica-based nanoparticle adheres to a mineral component of the osseous material of the subject, thereby increasing the volume of osseous material.

17. The method of claim 9, wherein the silica-based nanoparticle comprises a metallic core and a silicaceous shell disposed on the metallic core.

18. The method of claim 9, wherein the silica-based nanoparticle further comprises a polymeric protective coat.

19. The method of claim 18, wherein the protective coat is comprised of polyethylene glycol, polyvinyl pyrrolidone, PTMA, or PMP, or any combination thereof.

20. The method of claim 9, wherein the silica-based nanoparticle further comprises a label.

21. The method of claim 20, wherein the label is a fluorescent label.

22. A pharmaceutically acceptable composition comprising an effective dose of a silica-based nanoparticle, wherein the silica-based nanoparticle can increase the formation of osseous material in the subject.

23. The composition of claim 22, further comprising a carrier.

24. The composition of claim 22, wherein the silica-based nanoparticle increases the proliferation of a population of osteoblasts in the subject, thereby increasing the formation of osseous material.

25. The composition of claim 22, wherein the silica-based nanoparticle decreases the loss of osseous material from a bone of the subject.

26. The composition of claim 22, wherein the silica-based nanoparticle decreases the loss of osseous material from a bone of the subject by inhibiting the activity of osteoclasts in the subject.

27. The composition of claim 22, wherein the silica-based nanoparticle inhibits any of osteoclast activity in the subject by inhibiting an increase in a population of osteoclasts, inhibiting the differentiation of cells of a population of monocyte-macrophage cells into preosteoclasts, fusion of preosteoclasts into osteoclasts, or any combination thereof.

28. The composition of claim 22, wherein the silica-based nanoparticle decreases the loss of osseous material from a bone of the subject by inhibiting the ability of osteoclasts to remove osseous material from a bone of the subject.

29. The composition of claim 22, wherein the silica-based nanoparticle can adhere to a mineral component of the osseous material of the subject, thereby increasing the volume of osseous material.

30. The composition of claim 22, wherein the silica-based nanoparticle comprises a metallic core and a silicaceous shell disposed on the metallic core.

31. The method of claim 22, wherein the silica-based nanoparticle further comprises a polymeric protective coat.

32. The method of claim 31, wherein the protective coat is comprised of polyethylene glycol, polyvinyl pyrrolidone, PTMA, or PMP, or any combination thereof.

33. The composition of claim 22, wherein the silica-based nanoparticle further comprises a label.

34. The composition of claim 33, wherein the label is a fluorescent label.
